(19) United States
(12) Patent Application Publication
(10) Pub. No.: US 2002/0099174 A1
(43) Pub. Date: Jul. 25, 2002

(54) FILTRATION OF PLASMA MIXTURES USING CELLULOSE-BASED FILTER AIDS

(76) Inventors: ANNA JOHNSTON, PORT MELBOURNE (AU); JEFFERY RAYMOND DAVIES, IVANHOE (AU); PETER JAMES TURNER, LOWER PLENTY (AU); BRENTON JOHN WILKIE, NEW GISBORNE (AU)

Correspondence Address: KNOBBE MARTENS OLSON & BEAR LLP, 620 NEWPORT CENTER DRIVE, SIXTEENTH FLOOR, NEWPORT BEACH, CA, 92660, US

(21) Appl. No.: 09/142,604

(22) PCT Filed: Mar. 6, 1997
(86) PCT No.: PCT/AU97/00139

(30) Foreign Application Priority Data
Mar. 8, 1996 (AU) PN8585

(51) Int. Cl. C07K 1/00; C07K 14/00; C07K 16/00; C07K 17/00; A61K 35/14; A61K 38/16; A23J 1/00

(52) U.S. Cl. 530/364; 530/412; 530/414; 530/418; 530/419; 530/380; 530/829; 530/830; 530/831

(57) ABSTRACT

The present invention relates generally to a method of separating one or more components from a protein mixture. More particularly, this invention is directed to a method of separating one or more components of blood plasma comprising one or more filtration steps using a cellulose-based filter aid. The present invention is useful in the preparation of therapeutics, in particular plasma-based therapeutics for use in humans.

The present invention relates generally to a method of separating one or more components from a protein mixture. More particularly, this invention is directed to a method of separating one or more components of blood plasma comprising one or more filtration steps using a cellulose-based filter aid. The present invention is useful in the preparation of therapeutics, in particular plasma-based therapeutics for use in humans.

Throughout this specification, unless the context requires otherwise, the word “comprise”, or variations such as “comprises” or “comprising”, will be understood to imply the inclusion of a stated integer or group of integers but not the exclusion of any other integer or group of integers.

Bibliographic details of the publications referred to by author in this specification are collected at the end of the description.

Recent advances in the understanding of the function of blood plasma proteins and the deficiencies involved in a variety of blood disorders, combined with improvements in techniques for storage of the major protein components of human blood, have resulted in increased utilisation of specific sub-fractions of human blood, in particular the cellular components (erythrocytes, thrombocytes and leukocytes) and plasma protein fractions (albumins, fibrinogen and globulins including euglobulins, pseudoglobulins, α-globulin, β-globulin and γ-globulin), rather than whole blood, for therapeutic purposes.

The plasma protein fraction of human blood, in particular, is of enormous value to the pharmaceutical industry in the production of therapeutics for the treatment of fibrinogenic, fibrinolytic and coagulation disorders and immunodeficiencies, for example haemophilia, von Willebrand's disease and fibrinogen deficiency, amongst others. The major therapeutic fractions are: albumin, in several degrees of purity; immune serum globulin, both normal and specific; anti-haemophilic factor or factor VIII; prothrombin complex comprising factors II, VII, IX and X; and fibrinogen or factor I. The use of therapeutically-active plasma fractions eliminates the danger of hypervolemia and minimises the risk of contaminating proteins. Adequate replacement therapy for patients with coagulation disorders is only possible through the use of coagulation factor concentrates. Antibody titres high enough for prophylaxis or therapy can be achieved only through the use of immune serum globulin concentrates.

Many components of blood plasma, in particular the coagulation factors V and VIII and immune serum globulin are labile and must be prepared rapidly and carefully for maximum therapeutic efficacy and in order to minimise the risks associated with use of blood products For example, it is possible that partial denaturants of immune serum globulin, due to less-than-optimum fractionation procedures, may produce products which are toxic or highly immunogenic in recipients. Thus, there is a need in the plasma fractionation industry for improved fractionation methods which are more rapid, higher yielding, less denaturing and introduce few undesirable contaminants into the plasma fractions derived therefrom.

Cryoprecipitation is the first step in most methods in use today, for the large-scale production of plasma fractions. Fresh frozen plasma is pooled, thawed at below 5° C. and the precipitate is collected in continuous flow centrifuges (Guthohrleen and Falke, 1977; Avery, 1972). The supernatant fraction, known to those skilled in the art as a “cryosupernatant”, is generally retained for use.

The cryosupernatant may be used as a source of many plasma fractions, including fibrinogen, antithrombin III, prothrombin complex comprising the coagulation factors (II, VII, IX and X), albumin and immunoglobulin.

Subsequent processing of the cryosupernatant generally involves precipitation using organic precipitants such as ammonium sulfate, ethanol, acetone and polyethylene glycol. For example, in unique combinations of ethanol, subzero temperatures, pH, ionic strength and protein concentration, selective precipitation of plasma protein fraction occurs. These principles are fundamental to the Cohn Fractionation method (Cohn et al, 1946), which was developed to purify albumin.

With most organic precipitants, in particular cold ethanol, antithrombin III and fibrinogen are generally the first plasma proteins to precipitate. Albumin, euglobulin and lipoprotein are generally the last proteins to fractionate using cold ethanol.

Variations to the Cohn Fractionation have been developed by Oncley et al, (1949) to purify immunoglobulin from plasma. The Oncley method uses Cohn Fractions II and III as starting material and different combinations of cold ethanol, pH, temperature and protein concentrations to those described by Cohn et al (1946), to produce an active immune globulin serum fraction. Today, the Oncley method is the classic method used for production of immune serum globulin.

Selective separation of proteins, using ethanol or isoelectric precipitation, has also been exploited to further purify Cohn fractions. In particular, Curling (1980) used isoelectric precipitation to remove euglobulin protein from albumin-rich fractions derived from plasma.

Alternatively, or in addition, Cohn fractions may be further purified to remove solid-bound lipoproteins. Methods for the partitioning of lipoproteins into the solid phase are well-known to those skilled in the art and include, for example, the adsorption of lipoproteins to Aerosil™ or similar silicate material, amongst others.

In all precipitation steps used to fractionate plasma proteins, the solid and liquid phases must be separated. Usually, the large quantities of precipitate are pelleted by means of centrifugation. The protein precipitate, or paste, is removed from the centrifuge bowls and resuspended and further processed to other fractions, or the supernatant is collected and processed directly. The assortment of suitable centrifuges is limited, however, due to the exacting hygienic requirements and low operating temperatures required for plasma fractionation, in particular fractionation of labile proteins (eg: factor VIII). Unfortunately, the heat dissipation from a centrifuge is significant and places a large cooling requirement on the jackets. Centrifuges may need to be kept in process rooms which are kept at sub-zero temperatures to reduce the load on the machines. They often have insufficient solid holding capacity for large-scale processing and may require several bowl changes for each batch of plasma protein processed. Experience has shown that the maintenance requirements of centrifuges is high, leading to substantial down times, high maintenance costs and loss of capacity during repair. Furthermore, significant quantities of the mother liquor derived from the feed mixture are retained with the paste following centrifugation, which reduces yield of valuable proteins from the supernatant fraction and can lead to the presence of high levels of impurities in products derived from the paste, if washing of the paste is not performed.

An alternative to, or a procedure used in combination with, centrifugation, is the large-scale filtration of plasma proteins to remove precipitated. There are many types of filters that can be used to filter plasma fractions. The plate and frame filters are the easiest to use, have the lowest liquid volume to area ratio so heel volumes are minimal, and cake washing is very effective. Tubular filters are vertically orientated and are primarily used when the solid content is low, such as water purification. Rotating leaf filters are constructed with horizontal or vertical leaves in a vertical or horizontal chamber vessel. The horizontal leaves are used in intermittent operations as a polishing filter where solid loading is low and cycles times are long. The vertical leaves are designed for ease of cake removal, and when solid loading is high.

Filters may be used in combination with a filter aid to facilitate the flux during the filtration process. Filter aids are added to the solid-liquid mixture to prevent blinding/plugging of the filter mesh/support and to facilitate throughput during filtration by providing open channels for flow. The optimum filter aid is often one that gives the best clarity at the fastest flow. Essentially, a filter aid has to be highly permeable, have a good narrow particle/fibre size distribution, be chemically inert and physically robust.

Filter aids are used extensively in the process industry for various applications such as coal liquefaction (Jones et al, 1994; Shou et al, 1980), waste water treatment (Rudenko, 1981; Martin et al, 1993), food and beverage purification (Olsen et al, 1979; Hermia and Brocheton, 1994) and in the oil industry (Grichenko and El'shin, 1980; Soroka, 1975).

Cellulose filter aids are used widely in filtering plants where soluble silica from diatomaceous filter aids is undesirable, such as in the brewing, fermentation and metallurgical industries (see Dicalite™ Technical Bulletin, Rettenmaier & Sohn; Arbocel™ Technical Bulletin, Rettenmaier & Sohn). The utility of cellulose filter aids to the fractionation of pharmaceutical products, in particular blood products, remains undetermined.

In the plasma fractionation industry, filtration has been limited to the use of diatomaceous earth filter aids. Friedlie and coworkers from the Swiss Red Cross Blood Transfusion Service have explored filtration with diatomaceous earth filter aids (Perlite™ J-100, Celite™ 545 and Hyflo-Super-Cel™) in the production of albumin and gamma globulin from plasma fractionation. They concluded in early studies that whilst filtration of the crude fractions showed promising results, the adsorption effects of diatomaceous earth filter aids caused unacceptable protein loss in the separation of the purer fractions (Friedli et al, 1976). The Red Cross Blood Transfusion Service in Germany (Wolter, 1977) has trialed a vertical shank ZHF-S filter pre-coated with the diatomaceous earth filter aid, Hyflo Super-Cel™, for the isolation of albumin. Hao (1985), of the New York Blood Centre, also investigated Hyflo Super-cel™ as a filter aid in the filtration of Fraction IV-4 and found that albumin losses did, in fact, occur. De Jonge et al (1993) reported the use of the diatomaceous earth filter aid, Celite™, to filter out the Cohn precipitates, Fraction I, Fraction I+III, Fraction III, Fraction IV and Fraction V.

There are several problems associated with the use of diatomaceous earth filter aids, in the pharmaceutical industry, where high quality of the end-product is an essential pre-requisite. In particular, diatomaceous earth filter aids are extracted from the ground and undergo little pretreatment. Their quality is inherently variable depending on their source and they have to be acid washed if used in the pharmaceutical industry because of their prevalence to leaching heavy metals and aluminium. They are abrasive to pumps and electro polished surfaces.

Furthermore, with particular regard to the plasma filtration industry, where high quality and insolubility are important, diatomaceous earth filter aids activate the contact activation system, generating prekallikrein activator (PKA) and PKA complexes which can cause adverse clinical consequences.

In work leading up to the present invention, the inventors sought to develop new and better methods for the fractionation of protein mixtures, for example plasma protein mixtures, using filtration technology. In particular, the use of filter aids which are novel to the plasma fractionation industry has provided the means to develop a range of methods improved for the production of plasma protein fractions, such as Cohn fractions, albumin, lipoproteins and euglobulins.

Accordingly, one aspect of the present invention provides a method of separating a solid-phase material from a mixture of biomolecules comprising contacting said mixture with a cellulose-based filter aid to produce a slurry and passing or pumping said slurry through a filter vessel.

In an alternative embodiment, the present invention provides a method of separating a solid-phase material from a mixture of biomolecules, said method comprising the steps of:

(i) precoating a filter mesh with a cellulose-based filter aid; and

(ii) passing or pumping said mixture of biomolecules through the precoated filter mesh.

In addition to the use of the cellulose-based filter aid to pre-coat a filter mesh, additional filter aid may be added to the mixture of biomolecules. Accordingly, in a further alternative embodiment of the invention, additional cellulose-based filter aid is added to the mixture of biomolecules to form a slurry, and the slurry is passed or pumped through the precoated filter mesh.

According to the foregoing embodiments, the filtrate thus obtained may be optionally re-circulated back into the feed or filter vessel, until sufficiently clarified. The filtrate is then collected.

The flux rate used in any embodiment of the process described herein may be readily determined by those skilled in the art.

Preferably, one or more filter washes or flushings may be performed using a suitable solvent or aqueous buffer solution to wash the solid phase or filter cake obtained in order to remove residual mother liquor trapped therein, further increasing yields and improving the separation process. Each filter wash or flushing further displaces fluid within the filter vessel, thereby increasing the yield of a desired product in the filtrate.

The volume of buffer used in each filter wash or flushing may be readily determined by those skilled in the art.

Suitable wash solutions and conditions for this purpose will vary considerably depending upon the nature of the mixture of biomolecules and the stability of the desired end-product. Such conditions may be readily determined by those skilled in the art, without undue experimentation.

The present invention particularly extends to a method of separating a solid-phase material from a mixture of biomolecules wherein said mixture of biomolecules includes at least one plasma protein.

The cellulose-based filter aid may be any filter aid comprising cellulose as an active ingredient which functions to prevent plugging of the filter mesh or support or alternatively, or additionally, facilitates flow during the filtration process, is chemically-inert, physically-stable, non-abrasive and preferably does not leach aluminium.

The inventors have discovered that the optimum type and amount of a cellulose-based filter aid suitable for a particular filtration process will vary depending on the starting material to be filtered and the desired end-product. Generally, an optimum filter aid for a particular filtration will provide the fastest flow during filtration and produce a clearer filtrate than a sub-optimum filter aid. However, in order to obtain the best performance of the invention described herein, it is important that the filter not be overloaded, since overloading can result in any one or more adverse effects, including increased turbidity of filtrates, breakthrough and ultimately buckling of the filter meshing, filter damage, and bridging of the filter cakes between the filter meshes, amongst others.

Accordingly, a particularly preferred embodiment of the present invention provides for the invention to be performed using a maximum concentration of about 2.0% (w/v) cellulose-based filter aid (i.e. 20 grams cellulose-based filter aid per liter of solution) or 2.0% (w/w) cellulose-based filter aid (i.e. 20 grams cellulose-based filter aid per kg of plasma). Even more preferably, the cellulose-based filter aid is used at a concentration of about 0.5% (w/v) to about 2.0% (w/v) or alternatively, at a concentration of about 0.5% (w/w) to about 2.0% (w/w).

According to this preferred embodiment, the inventors have found that the addition of filter aid, in a concentration range of about 0.5% (w/v) to about 2.0% (w/v), to the feed mixture prior to filtration helps to provide a tight, porous bed that facilitates flow. Lower concentrations of filter aid than those stated herein have a tendency to either be insufficient to cast the filter meshes, thereby allowing the flow of solid-phase material through the filter mesh or alternatively, to result in a low porosity bed that incurs a high pressure drop and reduces the amount of feed mixture that is filterable. Concentrations of cellulose-based filter aid higher than those specifically stated herein result in a high porosity bed which allows too much solid-phase material in the feed mixture to flow through the filter bed, thereby reducing filtrate clarity, in addition to producing the problems identified supra.

Preferably, wherein a mixture of biomolecules includes at least one plasma protein derived from a plasma source and the desired end-product is a euglobulin-rich or lipoprotein-rich fraction, many cellulose-based filter aids may be appropriate including, for example Diacel™ 150, Diacel™ 200, Arbocel™ 200 or Vitacel™ 200, amongst others. As euglobulin or lipoprotein may be sequestered into the solid-phase material by being bound to fumed silica, for example by addition of Aerosil™ to Fraction I, the concentration of cellulose-based filter aid used in the filtration process, expressed as a percentage relative to the weight of fumed silica present in the feed mixture, must also be optimised to obtain the best performance.

Wherein the mixture of biomolecules includes at least one plasma protein derived from a plasma source and the desired end-product is an immunoglobulin-rich fraction, the preferred filter aid is a fine-grade cellulose, for example Diacel™ 150, amongst others.

Wherein the mixture of biomolecules is a plasma fraction which is substantially the same as fraction II+IIIW or fraction III obtained using the Cohn et al (1946) procedure or a modification thereof, it is preferred that the filter aid is a fine grade cellulose, for example Diacel™ 150, amongst others.

The cellulose-based filter aid may be pre-swollen in a suitable buffer or medium, preferably a buffer or medium which is iso-osmolar and at the same pH as the mixture of biomolecules. Alternatively, the cellulose-based filter aid may be added directly to the mixture of biomolecules and incubated for a time and under conditions sufficient to allow the filter aid to swell, prior to the step of passing or pumping the mixture through a filter mesh or filter vessel.

The swell time of the cellulose-based filter aid and conditions appropriate to allow swelling of same will be known to those skilled in the relevant art and will vary depending on the average particle diameter, temperature hydrophilicity, or ionic strength of the solution in which swelling is performed.

The present invention is adaptable to any filter vessel suitable for filtering a mixture of biomolecules such as a mixture of plasma proteins, for example a plate and frame filter, tubular filter or rotating leaf filter, amongst others. Plate and frame filters are the easiest to use, have the lowest liquid volume to area ratio so heel volumes are minimal, and cake washing is very effective. Plate and frame filters are amenable to filtration involving either pre-coating of the filter mesh or a recirculation method as hereinbefore defined. Tubular filters are vertically orientated and are primarily used when the solid content is low, such as water purification. Rotating leaf filters are constructed with horizontal or vertical leaves in a vertical or horizontal chamber vessel. The horizontal leaves are used in intermittent operations as a polishing filter where solid loading is low and cycles times are long. The vertical leaves are designed for ease of cake removal, and when solid loading is high. The inventors have found that, in the filtration of plasma protein mixtures, the recirculation method of filtration is preferred.

The method of filtration of the present invention is particularly useful in the separation of complex or simple mixtures of biomolecules, in particular complex or simple mixtures of plasma protein components.

The term “biomolecule” as used herein shall be taken to refer to any naturally-occurring or naturally-derived molecule including, but not limited to, amino acids, nucleotides, nucleosides, sugars, fats and polymers comprising same such as nucleic acids, proteins, peptides, polysaccharides, lipids and lipoproteins.

Accordingly, the term “mixture of biomolecules” as used herein extends to any mixture comprising more than one protein, nucleic acid, protein, peptide, polysaccharide, lipid, lipoprotein, amino acid, nucleotide, nucleoside, sugar or fat compound, which is derived from a biological source. In its present context, the term “mixture of biomolecules” extends to cell cultures, solutions of cells or sub-cellular components or cellular extracts, especially blood and blood-derived products such as plasma and fractions derived therefrom.

The term “solid-phase material” as used herein shall be taken to mean any compound, macromolecule or biomolecule in its solid form, irrespective of the means used to solidify said compound, macromolecule or biomolecule. For example, in the context of applications of the invention pertinent to the plasma fractionation industry, the term “solid-phase material” shall be taken to include both plasma protein precipitates produced and solid-bound lipoproteins, amongst others, derived from unfractionated or fractionated plasma or blood.

The present invention is not to be taken as being limited by any method or means used to produce a solid-phase material as hereinbefore defined, subject to the proviso that said method or means does not degrade the cellulose-based filter aid described herein.

Preferably, the solid-phase material is a biomolecule such as a plasma protein selected from the list comprising albumin, immunoglobulin, lipoprotein, euglobulin, factor VIII, prothrombin complex, antithrombin III or other components of blood, amongst others.

In the context of the present invention, the term “plasma protein” means a protein, polypeptide or peptide fragment derived from a plasma source which includes but is not limited to fresh-frozen plasma, non-fresh frozen plasma or a fraction thereof, such as an intermediate fraction produced using the fractionation schemes of Cohn et al (1946) or Oncley et al (1949) or a modification thereof or other plasma fraction. Accordingly, the term “plasma protein” is not to be taken as being limited to plasma fractions derived using ethanolic precipitation methods.

The term “derived from” as used herein shall be taken to indicate that a particular integer or group of integers has originated from a particular source as specified herein, but has not necessarily been obtained directly from that source. For example, a plasma protein may be derived directly from unfractionated blood, crude plasma or a fraction thereof.

A second aspect of the present invention is directed to a method of separation of a mixture of biomolecules comprising at least one filtration step using a cellulose-based filter aid.

In an alternative embodiment, the present invention provides a method of separation of a mixture of biomolecules comprising at least one filtration step using a cellulose-based filter aid, wherein said mixture of biomolecules contains at least one protein molecule.

In a particularly preferred embodiment, the present invention provides a method of separation of a mixture of biomolecules comprising at least one filtration step using a cellulose-based filter aid, wherein said mixture of biomolecules is blood plasma or a derivative thereof, such as but not limited to, a cryosupernatant, a resuspended plasma protein precipitate or an intermediate fraction associated with a Cohn or Oncley Fractionation Scheme, amongst others.

In a more particularly preferred embodiment, said blood plasma derivative fraction is any immunoglobulin-rich, euglobulin-rich or lipoprotein-rich fraction.

According to this embodiment of the present invention, an immunoglobulin-rich euglobulin-rich or lipoprotein-rich fraction is derived from fresh-frozen plasma or other plasma source or a derivative thereof, including a cryosupernatant or an intermediate fraction associated with the fractionation schemes of Cohn et al (1946) or Oncley et al (1949) or a modification thereof or other process known to those skilled in the art.

The person skilled in the art will be aware of the fractionation schemes of Cohn et al (1946) and Oncley et al (1949) and existing or suitable modifications thereof likely to produce a source of immunoglobulin, euglobulin or lipoprotein.

The term “cryosupernatant” as used herein will be known by those skilled in the art to refer to the supernatant fraction obtained from fresh-frozen plasma following thawing at a temperature below about 5° C., and centrifugation to remove the solid phase or cryoprecipitate.

In a further alternative embodiment, the present invention provides a method of separation of a mixture of biomolecules comprising at least one, preferably two, more preferably three and even more preferably four ethanol/acetate precipitation steps in which the solid and liquid phases produced therein are fractionated using at least one filtration step employing a cellulose-based filter aid.

In a particularly preferred embodiment said mixture of biomolecules comprises at least one blood plasma protein, for example lipoprotein, euglobulin, immunoglobulin, factor VIII protein, prothrombin complex, antithrombin III, or albumin, amongst others.

Accordingly, this embodiment of the invention is useful in the separation of solid and liquid phases of ethanol/acetate precipitation mixtures associated with plasma fractionation schemes, for example the Cohn Fractionation Scheme (Cohn et al, 1946) or the Oncley Fractionation Scheme (Oncley et al, 1949) and modifications thereof.

The methods described herein are particularly useful in the production of isolated biomolecules, in particular proteins derived from plasma, which are suitable for use as therapeutic reagents in the treatment or prophylaxis of clinical disorders.

A further aspect of the present invention provides an isolated biomolecule wherein at least one, preferably at least two and more preferably at least three steps in the isolation of said biomolecule involve the use of a cellulose-based filter aid to separate said biomolecule from other biomolecules in a simple or complex mixture of biomolecules.

Preferably said biomolecule is a protein, more preferably a therapeutic protein, even more preferably a human therapeutic protein, and even more preferably a human therapeutic protein derived from plasma, for example lipoprotein, euglobulin, immunoglobulin, factor VIII, prothrombin complex, antithrombin III, or albumin, amongst others.

In a most preferred embodiment, the present invention provides an isolated therapeutic plasma protein wherein at least one, preferably at least two and more preferably at least three steps in the isolation of said protein involve the use of a cellulose-based filter aid and wherein said protein is selected from the list comprising albumin, lipoprotein, immunoglobulin or euglobulin.

The products produced according to the method of the present invention are substantially free of undesirable contaminants. For example, it is particularly preferred that plasma proteins, in particular immunoglobulins and albumin, isolated according to the process described herein have low aluminium and PKA levels. Minimum acceptable aluminium and PKA level are defined by the Pharmacopoeia standards worldwide, such as the British Pharmacopoeia (BP), European Pharmacopoeia (EP) and/or United States Pharmacopoeia (USP) standards.

Wherein albumin is produced using the process of the present invention, it is particularly preferred that, in addition to low aluminium and low PKA, the level of lipoprotein in said isolated protein is below 3.0% (w/w), more preferably below 2.0% (w/w), even more preferably below 1.0% (w/w), still more preferably below 0.5% (w/w) and even still more preferably below 0.3% (w/w).

The present invention is further described in the following non-limiting Figures and Examples. The embodiments exemplified hereinafter are in no way to be taken as limiting the subject invention.

In the Figures:

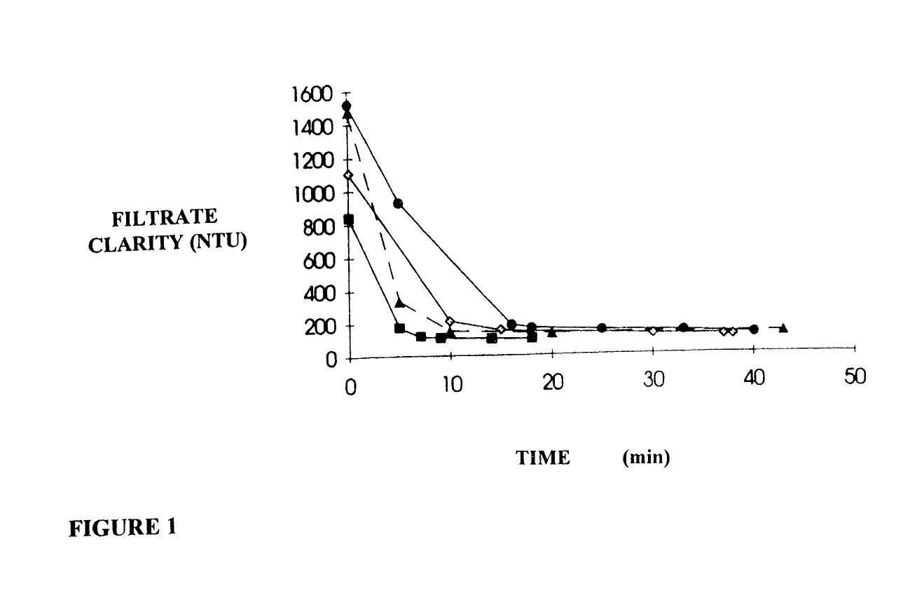

FIG. 1 is a graphical representation illustrating the improved clarity of filtrates derived from plasma protein Fraction I when using the cellulose-based filter aids Diacel™ 200 (closed circles; ●), Diacel™ 150 (closed squares; ▪) and Vitacel™ (open diamonds; ♦), compared to the diatomaceous filter-aid Celite™ (closed triangles; ▴).

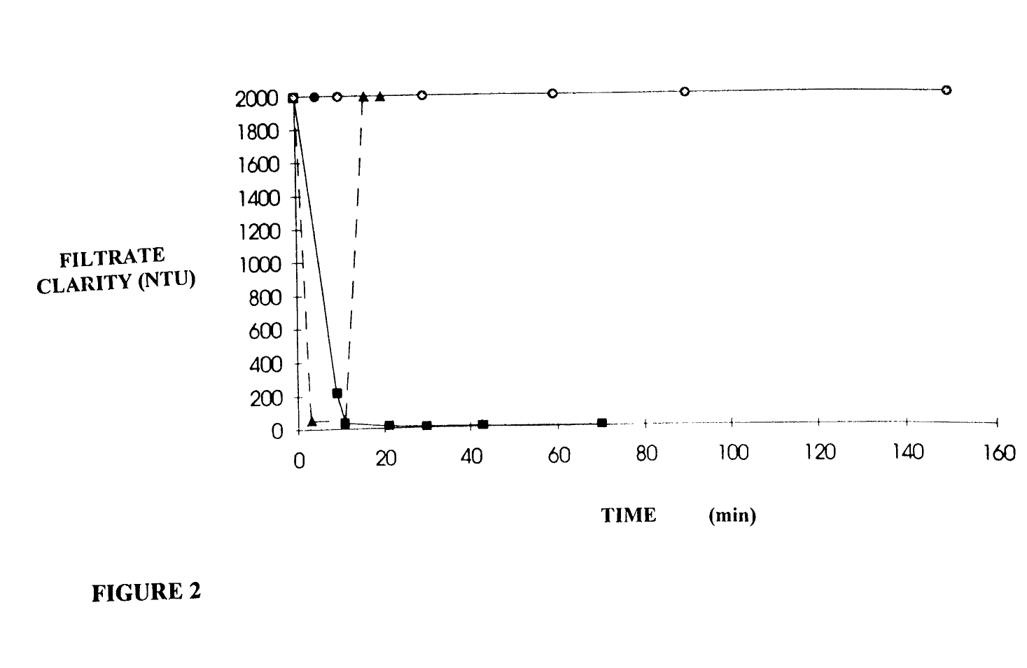

FIG. 2 is a graphical representation illustrating the improved clarity of filtrates derived from plasma protein Fraction II+IIIW mixture when using the cellulose-based filter aids Diacel™ 200 (closed circles; ●), Diacel™ 150 (closed squares; ▪) and Vitacel™ (open diamonds; ♦), compared to the diatomaceous filter-aid Celite™ (closed triangles; ▴).

### EXAMPLE 1

### Separation of Fraction I using Different Filter Aids

Four different pools of Fresh Frozen Plasma or cryosupernatant, batch size 200 Liters, were used in four independent runs using a pilot scale leaf filter, 2 m² in surface area. The starting material was cooled to ≦1° C., diluted with water for injections to give a protein concentration of 4% to 6% w/v. Sufficient ethanol and acetate buffer were then added to achieve the precipitating conditions of 8% v/v of ethanol and a final pH of 6.6 to 7.4. Under these conditions, and at −2° C., fibrinogen precipitated out leaving the immunoglobulin and albumin in solution. A cellulose based filter aid (Diacel™ 200, Diacel™ 150 or Vitacel™) was added to the Fraction I mixture at a ratio of 5 grams per liter of mixture and allowed to swell. The slurry was then pumped through the filter vessel and the filtrate recirculated back into the feed until clarity was achieved. The filtrate was collected until the feed was exhausted. A filter wash made up of 8% ethanol, 0.14M NaCl was then fed into the filter to wash the filter cake and to remove residual mother liquor derived from the feed mixture which is trapped in the filter cake. The heel volume of the filter was then blown out using precooled air or nitrogen, and finally the remaining liquid trapped in the vessel was drained out. The cake was then blown dry with precooled air or nitrogen. The filter was opened up, the cake was stripped from the filter meshes and discarded.

The filtrate clarity profiles of the various filter aids are presented in FIG. 1. The filtrate clarity obtained using a diatomaceous filter aid was also determined as a control. The finer grade of Diacel™ gave the steepest profile and the quickest throughput.

Table 1 compares the performance of the several runs of the various filter aids in the filtration of Fraction I and included in the table was the performance of the process centrifuges currently operating at Parkville. Generally the efficiency of the filtration separation was superior to that of the centrifuge separation, indicated by a higher filtrate clarity (low turbidity) and lower fibrinogen content in the filtrate. Protein yields were lower in the filtration runs than in the centrifugation runs because of the significant losses in dead volumes.

All the cellulose-based filter aids tested appeared to perform equally well, with clarities ranging from 82 NTU to 196 NTU. Diacel™ 150 required the least recirculation time, indicating that solids were trapped efficiently and quickly in the filter bed and the porosity of the bed was thus reduced quickly. However this filter aid also gave the greatest pressure drop as would be expected from this fine filter aid.

TABLE 1
Results from filtration of Fraction I mixture to yield Fraction I filtrate

| | NTU of | | Recirc. | protein yield | Fibrinogen |
|---|---|---|---|---|---|
| | filtrate | Pressure | time | in Fr I f/t | content |
| Filter aid | pool | (Bar) | (min) | g/L plasma | (mg/mL) |
| Vitacel ™ | 103 | 0.20 | 40 | 52.3 | not deter- |
| LC200 | | | | | mined |
| Celite ™ | 114 | 0.15 | 40 | 50.6 | not deter- |
| 580 | | | | | mined |
| Diacel ™ | 129 ± 59 | 0.4 ± 0.4 | 14 ± 5 | 49.8 ± 1.4 | <0.04 |
| 150 | (n = 3) | (n = 3) | | | |
| Diacel ™ | 116 ± 43 | 0.07 ± | 30 ± 18 | 52.6 ± 1.9 | <0.04 |
| 200 | (n = 3) | 0.03 | | | |
| Centrifuge | 520 | not appl. | not | 54-59 | 0.51 ± |
| | (n = 6) | | appl. | | 0.15 |
| | (314-1000) | | | | |

### EXAMPLE 2

### Separation of Fraction II+IIIW using Different Filter Aids

Pilot scale trials using a 2 m² leaf filter were run with batch sizes ranging from 15 to 18 kg of Fraction II+III cake. Frozen Fraction II+III cake, containing he filter aid from the previous fractionation step, was suspended in water for injections (at 0° C.) to give a protein concentration of 1% w/v. Sufficient ethanol and acetate buffer were then added to achieve the precipitating conditions of 20% v/v of ethanol and a final pH of 6.65. Under these conditions, and at −5° C., immunoglobulin reprecipitated leaving residual albumin and some lipoproteins in solution. The slurry was thoroughly mixed to ensure homogeneity of the Fraction II+IIIW mixture. No additional filter aid was added to this mixture. The slurry was then pumped through the filter vessel and the filtrate recirculated back into the feed until clarity was achieved. The filtrate was collected until the feed was exhausted. A filter wash made up of 20% ethanol was then fed into the filter to wash the filter cake and to remove residual mother liquor trapped in the filter cake. The heel volume of the filter was then blown out using precooled air or nitrogen, and finally the remaining liquid trapped in the vessel was drained out. The cake was stripped from the filter meshes and stored frozen until further processed into pure immunoglobulin.

The filtration of this mixture was only achieved using the finer grades of filter aid, Celite™ 580 and Diacel™ 150. Diacel™ 150 gave excellent filtration, and filtrate clarity was generally reached within 5 minutes. The run with Celite™ 580 was successful initially, with filtrate reaching 51 NTU after the first 5 minutes. However, once the pressure built up fine fibres were forced through the filter bed and leaked into the filtrate, contributing to the high turbidity of the filtrate pool of 988 NTU. Vitacel™ LC200 and Diacel™ 200 were unable to provide a tight enough filter bed to filter this Fraction II+IIIW mixture.

TABLE 2
Results from Fraction II + IIIW mixture

| | NTU of | Pressure | Recirc. time |
|---|---|---|---|
| Filter aid | filtrate pool | (Bar) | (min) |
| Vitacel ™ LC200 | >2000 | 2.0 | 145 |
| Celite ™ 580 | 988 | 2.0 | 5 |
| Diacel ™ 150 | 34 ± 25 | 0.6 ± 0.5 | 7 ± 5 |
| | (n = 3) | (n = 3) | (n = 3) |
| Diacel ™ 200 | >2000 | 0.7 ± 0.9 | >60 |
| Centrifuge | 55-915 | not appl. | not appl. |

### EXAMPLE 3

### Filtration Performances at Pilot Scale for Various Ratios of Cellulose-based Filter Aid

To determine the appropriate amount of cellulose-based filter aid in a typical separation, pilot scale trials were conducted to separate out albumin in the filtrate and immunoglobulin in the precipitate, using a 2 m² leaf filter to filter 250 kg to 500 kg of Fraction II+III mixture as starting material.

Varying amounts of Diacel™ 150 were added to the mixture, allowed to swell and then filtered using the same procedure outlined in Example 1, except that a 20% ethanol/NaCl filter wash was employed and the immunoglobulin-rich precipitate and the albumin-rich filtrate were both collected.

The filtration times, filtrate clarities and the pressure drop across the filter are presented in Table 3. As shown in Table 3, greater amounts of cellulose-based filter aid result in a decrease in the clarity of the filtrate obtained (i.e. higher turbidity). At the highest percentage of filter aid trialed, the pressure drop was the lowest, further indicating that the bed filter is more open to flow under these conditions. There appears to be little change in the filtration time for each amount of filter aid tested, however, at the highest percentage of filter aid tested, bridging of the filter cakes between the meshes was observed. Such bridging indicates the percentage of filter aid could not be increased beyond the 1.8% (w/v) level, without overloading of the filter and buckling of the filter meshes.

TABLE 3
A comparison of the filtration performance of Diacel ™ 150
at various concentrations

| Volume of | Amount of | | | |
|---|---|---|---|---|
| mix | filter aid | Time | NTU of | Pressure |
| (Liters) | (% w/v) | (hours) | filtrate pool | (Bar) |
| 285 (n = 1) | 0.8 | >6 | nd | 1.40 |
| 332 (n = 1) | 1.0 | 4.0 | 9.8 | 1.30 |
| 332 (n = 3) | 1.5 | 4.7 | 14.2 | 1.40 |
| 460 (n = 3) | 1.8 | 6.0 | 37.4 | 0.50 |

### EXAMPLE 4

### Removal of Lipoproteins and Euglobulin from an Albumin Rich Plasma Fraction

Approximately 80 liters of albumin rich plasma fraction was pH adjusted to 5.2 to precipitate euglobulins and then treated with fumed silica to absorb out lipoproteins. The filter aid Diacel™ 200 was then added to the mixture in a ratio of 2 gram of filter aid/gram of fumed silica and allowed to swell for at least 30 minutes. The mixture was then pumped into a plate and frame filter that had been fitted with filter pads and precoated with Diacel™ 200. The filtrate was collected the filter cake washed with a wash buffer, 5 mM sodium acetate, pH 5.2. The press was then dismantled and the cake discarded.

A similar run was performed using a pilot scale leaf filter, 2 m², with a batch size of 160 Liters. In this case the filter was not precoated, since the high flux achievable on the filter ensured that the filter aid, Diacel™ 200, was distributed homogenously on the filter meshes. The filtrate was recirculated until clear before collecting. The cake was then washed with wash buffer, the heel volume collected and the filter cake removed and discarded.

Table 4 compares the performance of the two filters.

TABLE 4
Filter performance of Diacel ™ 200 in the
euglobulin/lipoprotein removal step

| Parameter | Plate and frame | Leaf filter |
|---|---|---|
| Clarity at start of run (NTU) | 71 | 57 |
| Clarity at the end (NTU) | 16 | <18 |
| Clarity of final pool (NTU) | 26 | 10 |
| Filtration time (min) | 40 | 100 |
| Lipoprotein content in feed (% w/w) | 2.9 | 3.1 |
| Lipoprotein content of filtrate (% w/w) | 0.2 | 0.3 |
| Protein recovery (% w/w) | 85 | 82 |

The filtration performance of Diacel™ 200 works equally well in both filters, with the runs giving high filtrate clarity (low turbidity) and an equal recovery of protein (one would not expect 100% recovery because the precipitation has removed some protein). Reduction of lipoprotein in both runs is at least ten fold reflecting the performance of both the plate and frame and the leaf filter. The filtration time in the second run is much greater than the first run reflecting the doubling in batch size.

### EXAMPLE 5

### A Comparison of the Separation of Fraction III using Centrifugation and Filtration at Production Scale

A production scale leaf filter (18 m²) was commissioned and validated for the filtration of Fraction I through to Fraction III filtrate. The results reported here summarise the data from three runs of the final filtration of Fraction III mixture. Each run was performed as follows.

A 400 kg batch of Faction II+IIIW cake, which contained Diacel™ 150 from a previous Cohn Fraction step, was resuspended in water for injection at 0° C. to achieve a protein content of approximately 1% w/v. Sufficient ethanol and acetate buffer were then added to achieve the precipitating conditions of 17% v/v of ethanol and a final pH of 5.35. Under these conditions, and at −5° C. immunoglobulin M, immunoglobulin A and several lipid proteins remained precipitated, whilst immunoglobulin G resolubilised. The mixture was then pumped through the filter vessel and the filtrate recirculated back into the feed until clarity was achieved. The filtrate was collected until the feed was exhausted. A filter wash made up of 17% v/v ethanol, 0.015M sodium acetate was then fed into the filter to wash the filter cake and to remove residual mother liquor trapped in the filter cake. The heel volume to the filter was then blown out using precooled air or nitrogen, and finally the remaining liquid trapped in the vessel was drained out. The cake was then blown dry with precooled air or nitrogen. The filter was opened up, the cake was stripped from the filter meshes and discarded. Samples of Fraction III filtrate were taken and characterised. The filtrate was then pH adjusted to 4.0, concentrated up to 7% v/w protein and then diafiltered against water for injections, before being formulated in maltose. Samples of the final product were also characterised to detect trace levels of other plasma proteins. Table 5 summarises the characterisation of the filtrate and final products from these three runs, and compares them with data from production centrifuges.

TABLE 5
Fraction III filtrate and final product characterisation

| | Production | Production |
|---|---|---|
| | centrifuges | filters |
| clarity, NTU in supernatant/filtrate | 11.8 ± 7.5 | 1.4 ± 0.2 |
| IgM in final product, mg/mL | 0.05 ± 0.01 | 0.09 ± 0.2 |
| IgA in final product, mg/mL | 0.17 ± 0.03 | 0.19 ± 0.05 |
| plasminogen in final product, μg/mL | 0.52 ± 0.36 | 0.33 ± 0.07 |

Table 5 illustrates that the filtrates from the filters are clearer than the supernatant from the centrifuges with lower NTU values. The supernatant III from the centrifuges were subsequently polish filtered through a plate and frame filer to achieve the same level of clarity. By comparing the levels of IgM, IgA and plasminogen in the final products (these plasma proteins remain precipitated at this step in the Cohn fractionation scheme) it is apparent that the filtration as opposed to centrifugation of this Fraction III mixture has not compromised product quality.

### EXAMPLE 6

### Filtration Performances at Production Scale for Various Ratios of Cellulose-based Filter Aid

Two production-scale filters (18 m²) were used to filter Fraction II+III mixtures, using the same procedure outlined in Example 1, except that a 20% ethanol/NaCl filter wash was employed and the immunoglobulin-rich precipitate and the albumin-rich filtrate were both collected. The amount of filter aid (% w/v) was progressively reduced, in order to maximise the space available within each filter.

The results are shown in Table 6. Data shown in Table 6 were obtained from two different-sized batches of Faction II+III, derived from 2500 kg and 5000 kg of plasma.

As shown in Table 6, the greater the percentage of filter aid used, the faster the filtration time (i.e. faster flow). At the production scale tested, the filtrate clarity is more carefully controlled than at the pilot scale and, as a consequence, the filtrate clarities appear lower. The greater the amount of filter aid, the greater is the mass of precipitate collected, which reduces the effective capacity of each filter for the same mixture volume.

TABLE 6
A comparison of the filtration performance at production scale for
various concentrations of cellulose-based filter aid (n = 3)

| Volume of | Amount of | | | Mass of |
|---|---|---|---|---|
| mix | filter aid | Time | NTU of | precipitate |
| (Liters) | (% w/v) | (hours) | filtrate pool | (kg) |
| 3600 | 1.5 | 4.09 | 6.6 | 254 |
| 3700 | 1.1 | 6.30 | 6.5 | 213 |
| 7100* | 1.5 | 5.24 | 4.9 | 2 × 254 |
| 7200* | 1.0 | 7.20 | 5.2 | 2 × 224 |
| 7000* | 1.0 | 8.00 | 7.2 | 2 × 202 |

*Two filters were utilised and half of the total volume was processed in parallel through each filter.

### EXAMPLE 7

### Yields of Immunoglobulin from a Production-scale Filtration Process

Production-scale filters were used to filter Cohn precipitated mixtures in the manufacture of immunoglobulin. At each stage, Fraction I mixture, Fraction II+III mixture, Fraction II+IIIW mixture or Fraction III mixture were filtered, using 1.5 or 2.0 filter vessel volumes (7500 kg or 10,000 kg) of filter wash to recover the filtrate from the vessel.

Data shown in Table 7 compare the yields of immunoglobulin G at three different batch sizes, when either 1.5 or 2.0 filter vessel volumes of filter wash were used. The data indicate that at both of the high production scales tested, yields are significantly higher when higher filter wash volumes are employed.

TABLE 7
A comparison of the yields from the production facility for different
batch sizes and different filter wash volumes

| Mass of Plasma | | |
|---|---|---|
| Processes (MPP) | Filter Wash Volume | Yield |
| (kg) | (×MPP) | (g per kg plasma) |
| 2,500 | 1.5 | 3.60 |
| 5,000 | 2.0 | 4.09 |
| 7,500 | 2.0 | 4.14 |

### EXAMPLE 8

### Assessment of Product Quality

Aluminium and PKA levels were determined for immunoglobulin and albumin fractions produced using a cellulose-based filter aid as described herein. Results are indicated in Tables 8 and 9. In Table 8, data indicate that the albumin quality is not compromised by exposure to cellulose-based filter aid and that the product produced using the cellulose-based filter aid as described herein is low in PKA and PKA complexes and has a low aluminium level. In Table 9, data indicate that filtration using a cellulose-based filter aid has no detrimental effect upon the characteristics of the final product, as measured by the low aluminum content, low kallikrein and low PKA content of immunoglobulin preparations produced according to the invention.

TABLE 8
Characterisation of albumin (production scale)

| Contaminant | Pharmacopoeia Limit | Result (n = 5) |
|---|---|---|
| Aluminium | <100 μg/L | 8 ± 4 μg/L |
| PKA-C₁ esterase | ≦35 IU/mL | <7 IU/mL ± 3 IU/mL |
| PKA | ≦28.6 IU/mL | 4.8 IU/mL ± 1.3 IU/mL |

TABLE 9
Characterisation of IgG obtained from filtration using cellulose-based
filter compared to IgG obtained using a centrifugation
process aid (production scale).

| Contaminant | Centrifugation (n = 3) | Filtration (n = 3) |
|---|---|---|
| Aluminium (μg/L) | 67 μg/L ± 21 μg/L | 34 μg/L ± 3 μg/L |
| Kallikrein (% std reference) | 453 ± 147 | 167 ± 33 |
| PKA (IU/mL) | <1.0 IU/mL | <1.0 IU/mL |

Those skilled in the art will appreciate that the invention described herein is susceptible to variations and modifications other than those specifically described. It is to be understood that the invention includes all such variations and modifications. The invention also includes all of the steps, features, compositions and compounds referred to in the specification, individually or collectively, and any and all combinations or any two or more of said steps or features.

### REFERENCES

Avery C. G., Fractionation of blood by centrifugation. Process Biochem., 7.20, 1972

Cohn E. J., Strong L. E., Hughes L. E., Mulford D. J., Ashworth J. N., Melin M. and Taylor H. L., Preparation and Properties of Serum and plasma fractions. J. American Chemical Society, 68, 459, 1946

Curling J. M., Methods of Plasma Protein Fractionation, Ed. J. M. Curling, Academic Press, London U.K., 1980

De Jonge E., M. A. W. van Leeuwen, H. Radema, P. H. J. M. Linssen and J. Over, Filtration processes in the Cohn fractionation. Biotechn. Blood Proteins, Eds C. Rivat, J.-F. Stoltz. Colloque INSERM, 227, 49-54, 1993

Dwyer, J. L., Filtration in the food, beverage and pharmaceutical industries. In Clyde Orr Ed., Filtration Principles and Practices., Part II. Marcel Dekker, NY. pp 121-199, 1979

Friedli H., Mauerhofer M., Faes F. and Kistler P. Studies on New Process Procedures in Plasma Fractionation on an Industrial Scale, Vox Sang., 31, 389-295, 1976

Grichenko, A. A.; El'shin, A. I. Use of Adsorbents To Increase Filtration Rate Of Vnii Np-360. Chemistry and Technology of Fuels and Oils (English translation of Khimiya i Tekhnologiya Topliv i Masel), 16 (9-10), 567-569, 1980

Hermia, J.; Brocheton, S. Comparison of modern beer filters. Filtration and Separation 31 (7), 721-725, 1994

Hao, Y, Pilot scale Preparation of Human Serum Albumin, Vox Sang., 49, 1-8, 1985

Jones, M. A.; Kimber, G. M.; Pass, R-; Romey, I. Evaluation of cokes as filter aids in direct coal liquefaction. International Journal of Energy Research, 18 (2) 287-297, 1994

Kistler P. and Nitschmann H. Large scale production of human plasma fractions, Vox Sang., 7, 414-424, 1962

Martin, H. Lee; Norford, Stephen W.; Diener, Glenn A. Metal finishing wastewater pressure filter optimization Proceedings of the AESF Annual Technical Conference 1993. Publ by American Electroplaters & Surface Finishers Soc Inc, Orlando, Fla., USA. p 559-562, 1993

Olsen, B. Winstrom; Madsen, R. F.; Nielsen, W. Kofod. Sugar Beet Phenols—2. Investigation Of Phenolic Compounds From Sugar Beet in Relation to The Formation Of Colour. International Sugar Journal, 81 (972) 362-367, 1979

Oncley, J. L., Melin, M., Richert, D. A., Cameron J. W., and Gross, P. M., Jr., The separation of antibodies, isoagglutins, prothrombin, plasminogen, and β-1-lipoprotein into subfractions of human plasma. J. Am. Chem. Soc., 79, 4666-4671, 1949

Reti A. R., The Design and Use of Filtration Systems, in Methods of Plasma Protein Fractionation, Academic Press, Edt. J. M. Curling, pg 291-304, 1980

Rudenko, L. I.; Sklyar, V. Ya. Composite Filter Aids For Cleanup Of Additives. Chemistry and Technology of Fuels and Oils (English translation of Khimiya i Tekhnologiya Topliv i Masel), 19 (7-8) 384-386, 1983

Schneider-W; Wolter-D; McCarty-L J. Technical improvements in heat-ethanol isolation of serum albumin. Blut. 33(4), 275-80, 1976

Shou, J. K.; Collins, D. M.; Do, D. M.; Scharff, R. P. Precoat Filtration of Coal Liquid Feasibility Study Of Bottom Ash Precoat. Separation Science and Technology, 15 (3), 201-221, 1980

Soroka, A. S. Use of Filter-Aid Powders In Removing Solid Contaminants From Intermediate Products In Manufacture Of Succinimide Additives. Chemistry and Technology of Fuels and Oils (English translation of Khimiya i Tekhnologiya Topliv i Masel), 11 (9-10), 780-783, 1975

Wolter D., The use of coarse filtration for Separation of Plasma Fractions, International Workshop on Technology for Protein Separation and Improvement of Blood Plasma Fractionation, Edt. H. E. Sandberg, Virginia Sept., pg 129, 1977.

1. A method of separating a solid-phase material from a mixture of biomolecules comprising contacting said mixture with a cellulose-based filter aid to produce a slurry and passing or pumping said slurry through a filter vessel or filter mesh to obtain a filtrate and filter cake.

2. A method of separating a solid-phase material from a mixture of biomolecules, said method comprising the steps of:
(i) precoating a filter mesh with a cellulose-based filter aid; and
(ii) passing or pumping said mixture of biomolecules through the precoated filter mesh to obtain a filtrate and a filter cake.

3. The method according to claim 2, wherein additional cellulose-based filter aid is added to the mixture of biomolecules prior to the step of passing or pumping said mixture of biomolecules through the precoated filter mesh.

4. The method according to any one of claims 1 to 3, comprising the further step of re-circulating the filtrate one or more times, into the feed or filter vessel.

5. The method according to any one of claims 1 to 4, comprising the further step of washing or flushing the filter cake with a suitable solvent or aqueous buffer solution to remove residual mother liquor derived from the feed mixture.

6. The method according to claim 5, wherein the step of washing or flushing the filter cake involves up to three filter vessel volumes of the solvent or aqueous buffer solution.

7. The method according to any one of claims 1 to 6, wherein the concentration of cellulose-based filter aid is up to about 2.0% (weight per unit volume of feed mixture or weight per unit weight of feed mixture).

8. The method according to claim 7, wherein the concentration of cellulose-based filter aid is in the range of about 0.5% to about 2.0% (weight per unit volume of feed mixture or weight per unit weight of feed mixture).

9. The method according to any one of claims 1 to 8, wherein the mixture of biomolecules is selected from the list comprising blood, fresh-frozen plasma, non-fresh frozen plasma, cryosupernatant or plasma fraction derived therefrom, such as an intermediate Cohn fraction or Oncley fraction, or other plasma fraction.

10. The method according to claim 9 wherein the solid-phase material is a plasma protein selected from the list comprising albumin, immunoglobulin, lipoprotein, euglobulin, factor VIII, prothrombin complex, antithrombin III or other components of blood, amongst others, in a precipitated or complexed or aggregated form or bound to an insoluble carrier such as, but not limited to, fumed silica.

11. The method according to claim 10, wherein the cellulose-based filter aid possesses one or more of the characteristics:
(i) it facilitates the flow of the feed mixture through the filter mesh during the filtration process;
(iii) it does not generates PKA levels in plasma-derived products which are above BP, EP or USP standards; and
(iv) it does not leach aluminium into plasma-derived products at levels which are above BP, EP or USP standards.

12. The method according to claim 11, wherein the cellulose-based filter aid is selected from the list comprising Diacel™ 150, Diacel™ 200, Arbocel™ 200 or Vitacel™ 200, amongst others.

13. A method of separation of a solid-phase material from blood, fresh-frozen plasma, non-fresh frozen plasma, cryosupernatant or plasma fraction derived therefrom, such as an intermediate Cohn fraction or Oncley fraction, or other plasma fraction, said method comprising at least one filtration step to recover said solid-phase material, wherein said filtration step is employed using a cellulose-based filter aid.

14. The method according to claim 13, wherein the solid-phase material is a plasma protein selected from the list comprising albumin, immunoglobulin, lipoprotein, euglobulin, factor VIII, prothrombin complex, antithrombin III or other components of blood, amongst others, in a precipitated or complexed or aggregated form or bound to an insoluble carrier such as, but not limited to, fumed silica.

15. The method according to claim 13 or 14, preceded by at least one ethanol/acetate precipitation or treatment with fumed silica to produce the solid phase material.

16. An isolated biomolecule produced according to the method defined by any one of claims 1 to 15.

17. The isolated biomolecule according to claim 16, further characterised as a protein for therapeutic or prophylactic treatment of humans or mammals.

18. The isolated biomolecule according to claim 16 or 17, derived from blood, fresh-frozen plasma, non-fresh frozen plasma, cryosupernatant or plasma fraction derived therefrom, such as an intermediate Cohn fraction or Oncley fraction, or other plasma fraction.

19. The isolated biomolecule according to claim 18, selected from the list comprising lipoprotein, euglobulin, immunoglobulin, factor VIII, prothrombin complex, antithrombin III, or albumin, amongst others.

20. The isolated biomolecule according to claim 19, selected from the list comprising albumin, lipoprotein, immunoglobulin or euglobulin.

21. The isolated biomolecule according to claim 20, comprising albumin.

22. The isolated biomolecule according to claim 21, wherein the level of lipoprotein therein is below about 3.0% on a weight-for weight (albumin/lipoprotein) basis.

23. The isolated biomolecule according to any one of claims 16 to 22, wherein the level of PKA and/or aluminium is a preparation of said biomolecule is low.

24. The isolated biomolecule according to claim 23, wherein the level of PKA and/or PKA-C₁ esterase and/or kallikrein and/or aluminium is a preparation of said biomolecule is below the BP, EP or USP minimum standard.

25. The isolated biomolecule according to claim 24, wherein the level of aluminium in immunoglobulin preparations is below about 50 ug/L.

26. The isolated biomolecule according to claim 24, wherein the level of aluminium in albumin preparations is below about 10 ug/L.

27. The isolated biomolecule according to claim 24, wherein the level of PKA in albumin is below about 5 IU/mL.

28. The isolated biomolecule according to claim 24, wherein the level of PKA-C₁ esterase in albumin preparations is below about 10 IU/mL.
